(19) United States
(12) Patent Application Publication
(10) Pub. No.: US 2010/0030024 A1
(43) Pub. Date: Feb. 4, 2010

(54) DIAGNOSIS OR INTERVENTION INSIDE THE BODY OF A PATIENT USING A CAPSULE ENDOSCOPE

(76) Inventor: Sven Sitte, Erlangen (DE)

Correspondence Address: BRINKS HOFER GILSON & LIONE, P.O. BOX 10395, CHICAGO, IL, 60610, US

(21) Appl. No.: 12/508,114

(22) Filed: Jul. 23, 2009

(30) Foreign Application Priority Data
Jul. 28, 2008 (DE) 10 2008 035 092.3

(51) Int. Cl. A61B 1/00

(52) U.S. Cl. 600/118

(57) ABSTRACT

An apparatus for carrying out a minimally invasive diagnosis or intervention inside the body of a patient is provided. The apparatus includes a capsule endoscope, which can be introduced into the body of the patient and includes at least one medical instrument. At least one transmission antenna for emitting electromagnetic radiation is arranged outside the body. At least one reception antenna for receiving the electromagnetic radiation is provided in or on the capsule endoscope. The current position of the capsule endoscope is calculated in an analysis unit using antenna signals generated by the interaction of the transmission antenna with the reception antenna.

The present patent document claims the benefit of German Patent Application No. DE 10 2008 035 092.3, filed on Jul. 28, 2008, which is hereby incorporated by reference.

### BACKGROUND

The present embodiments relate to a medical apparatus for carrying out a minimally invasive diagnosis or intervention inside the body of a patient using a capsule endoscope.

A medical apparatus having a capsule endoscope is disclosed in DE 101 42 253 C1. The capsule endoscope has an ellipsoidal housing and can be introduced into the body of the patient. The capsule endoscope includes at least one medical instrument.

This medical instrument can be designed as a diagnostic instrument for determining measurement data. The diagnostic instrument is designed as an imaging system. The housing of the capsule endoscope includes a miniaturized video camera. Using this video camera, it is possible to record diagnostic images of a body region inside the body of the patient.

The medical instrument can also be designed to carry out a medical intervention. This intervention can be the removal of a tissue sample from a body region, for example. However, the medical intervention can also be the release of a drug inside the body of the patient, or similar. Such a medical intervention can be carried out with reduced stress to the organism of the patient.

The capsule endoscope can also be designed to include a plurality of medical instruments.

For the purpose of carrying out an effective medical diagnosis or intervention, the current position of the capsule endoscope during the diagnosis or intervention is important. The current position includes the spatial position of the capsule endoscope and the orientation in the space of the capsule endoscope.

### SUMMARY AND DESCRIPTION

The present embodiments may obviate one or more of the drawbacks or limitations inherent in the related art. For example, in one embodiment, the current position of the capsule endoscope inside the body of the patient, while carrying out a minimally invasive diagnosis or intervention, is determined with sufficient accuracy.

In one embodiment, at least one transmission antenna for emitting electromagnetic radiation is arranged outside the body of the patient. A power amplifier, such as those used in audio systems, can be used for amplifying the electromagnetic radiation emitted by the at least one transmission antenna. At least one reception antenna for receiving the electromagnetic radiation is provided in or on the capsule endoscope. The at least one reception antenna is provided inside the housing of the capsule endoscope or attached to the housing. The current position of the capsule endoscope inside the body is determined by an analysis unit on the basis of the antenna signal that is induced by the interaction of the transmission antenna with the reception antenna, or the induced antenna signals. In The signal amplitudes that are generated in the transmission antenna and/or in the reception antenna are analyzed as antenna signals for determining the current position. An amplitude signal may be proportional to the electrical field strength received by the respective antenna. Accordingly, the current position of the capsule endoscope can be identified at all times during the medical investigation or intervention. Measurement data, such as diagnostic images, may be assigned to the current position of the capsule endoscope while the data is captured. In the case of a medical intervention, it is possible to ensure that this takes place at the desired location. Furthermore, it is possible to display the current position of the capsule endoscope on a display unit, such as a computer monitor, while carrying out the minimally invasive diagnosis or intervention. The position at which the capsule endoscope is located inside the body is clear (e.g., displayed) for an examining doctor.

In one embodiment, the electromagnetic radiation has a frequency of approximately 30 kHz. Radiation in the range of 30 kHz is attenuated and delayed to approximately the same extent by different tissue types. Depending on the position of the capsule endoscope inside the body, this ensures that there are no, or only slight, tissue-dependent propagation time differences in the antenna signals at the location of the transmission antenna or the reception antenna. The current position of the capsule endoscope may be determined with little error.

In one embodiment, the capsule endoscope features a communication unit for communicating with a control unit or regulating unit which is situated outside the body. The communication unit may transfer measured values, such as diagnostic images, from the capsule endoscope to the control unit. The diagnostic images are processed by the control unit. The diagnostic images may be displayed directly on a display unit, such as a monitor, which is assigned to the control unit. However, the diagnostic images can also be stored on a data storage facility which is assigned to the control unit, for the purpose of subsequent reporting. In addition, control signals transmitted by the control unit can be received by the communication unit. The control signals effectively allow remote control of the at least one medical instrument. Selective actions of the medical instrument may be triggered when a desired position of the capsule endoscope has been reached. For example, camera settings can be changed remotely in the case of a diagnostic instrument which is designed as a video camera. This may relate to the camera focus of the video camera or to the illuminance of a lighting unit assigned to the video camera, for example.

In the case of a correspondingly designed medical instrument, the removal of a tissue sample can be carried out under remote control. If the medical instrument is designed to release a drug inside the body, this drug may be released under remote control.

In one exemplary embodiment, a carrier frequency of approximately 400 MHz is provided for communication between the communication unit and the control unit. By virtue of such a carrier frequency, high bandwidths can be transferred using low power consumption. Large quantities of data, such as the data generated for diagnostic images of a video camera, may be transferred quickly. The transfer may take place so quickly that a moving realtime image can be sent from the location of the capsule endoscope. The different attenuation and delay of the carrier frequency in different tissue types is not important for the transfer of data. The low power consumption additionally ensures that the capsule endoscope does not overheat. Any burning inside the body of the patient is prevented.

Measurement data that has been measured by the capsule endoscope may be associated with the relevant current position of the capsule endoscope by the analysis unit. Consequently, even long after the actual investigation, measurement data that was measured during the investigation, such as diagnostic images, can still be assigned unambiguously to its recording location inside the body. The diagnostic images can be evaluated any number of times in the form of an image sequence or film, even after the investigation.

In one embodiment, the at least one transmission antenna is configured to generate an inhomogeneous electromagnetic field. In this context, the term “inhomogeneous” includes both a spatial and a temporal change of the electromagnetic field. The electromagnetic field derives from the totality of the electromagnetic radiation emitted by the at least one transmission antenna. Antenna signals are generated in the at least one reception antenna depending on the location of the capsule endoscope. The current position of the capsule endoscope can be identified with reference to this at least one antenna signal.

Provision may be made for a plurality of transmission antennas having orientation vectors that are linearly independent relative to each other. The orientation vectors may be orthogonal relative to each other. Accordingly, the electromagnetic field can be specified within a wide framework. An inhomo-geneous electromagnetic field is easy to generate in this way.

The transmission antennas may be configured to emit the electromagnetic radiation alternately. In other words, time multiplexing is provided with regard to the operation of the transmission antennas. As a result of this alternating operation, an inhomogeneous electromagnetic field is produced even if the distance of all transmission antennas from the capsule endoscope is the same and if the transmission power of the electromagnetic radiation is the same. The individual power supplies of the transmission antennas are operated in a timed manner for this purpose, such that the individual transmission antennas are switched on or switched off in each case.

The timing may be preset such that one transmission antenna is switched on in each case and all other transmission antennas are switched off. Antenna signals which correlate with the electromagnetic field that is generated by a specific transmission antenna are therefore produced in chronological sequence at the location of the at least one reception antenna. Given a known electromagnetic field of the transmission antennas, it is therefore possible to calculate the current position of the capsule endoscope with reference to the different antenna signals.

A plurality of reception antennas having orientation vectors which are linearly independent relative to each other are arranged in or on the capsule endoscope. When using three reception antennas in particular, the whole inhomogeneous electromagnetic field in all three spatial directions may be captured by the reception antennas.

A three-dimensional inhomogeneous magnetic field may be generated by three transmission antennas having linearly independent orientation vectors. This inhomogeneous magnetic field interacts in all three spatial directions at the location of the capsule endoscope with three reception antennas having linearly independent orientation vectors.

The analysis unit is assigned to the capsule endoscope. The antenna signals generated in the reception antennas are analyzed at the location of the capsule endoscope. The communication unit is configured to send the current position, which is determined by the analysis unit, to the control unit. In this case, the analysis unit may be designed in such a way that the amplitude signals are captured and prepared for the transfer by the communication unit. The current position of the capsule endoscope is determined in the control unit in this case.

If the capsule endoscope measures measurement data, such as diagnostic images, the measurement data may be supplemented, at the location of the capsule endoscope, with the current position. As a result, further processing of the data in the control unit is no longer necessary. Instead, the measurement data is unambiguously supplemented with the current position of the capsule endoscope as a location stamp.

In one embodiment, the at least one reception antenna can be loaded and unloaded alternately in order to generate varying antenna signals in the at least one transmission antenna. The analysis unit is assigned to the at least one transmission antenna and determines the current position of the capsule endoscope from the varying antenna signals of the transmission antenna.

The capsule endoscope may include an energy storage unit for storing the electrical energy that is transmitted with the electromagnetic radiation. The electromagnetic radiation, which is emitted for the purpose of localizing the capsule endoscope, may supply the capsule endoscope with electrical energy at the same time. The energy storage unit takes the form of an accumulator or capacitor. Charging the energy storage unit before using the capsule endoscope is unnecessary. The maintenance required for such a capsule endoscope is reduced accordingly. After use, the capsule endoscope need only be thoroughly cleaned or sterilized. An opening for the purpose of exchanging the energy storage unit, such as a battery, is unnecessary. If the charging process of the energy storage unit is carried out intermittently instead of continuously, the at least one reception antenna is alternately loaded and unloaded.

In one embodiment, the at least one transmission antenna is arranged on a wall of a treatment room containing the apparatus. The transmission antenna is attached to a mounting on the wall, for example. A transmission antenna in the form of a rod antenna may be installed in the treatment room in a space-saving manner. The permanent installation on the wall to a large extent prevents any accidental contact with one of the transmission antennas, for example, by operating staff. The electromagnetic radiation emitted by the transmission antennas is not adversely affected by any such disruptions. A high level of accuracy is achieved when analyzing the antenna signals at the location of the transmission antennas or the reception antennas. Accordingly, a high level of accuracy is achieved when identifying the current position of the capsule endoscope. Directive efficiency for the transmission antenna is provided by a wall mounting. This directive efficiency results in amplified interaction with the at least one reception antenna and hence to higher antenna signals for the analysis.

If the capsule endoscope is supplied with electrical energy via its at least one reception antenna, the supply of electrical energy to the capsule endoscope is also improved by virtue of the higher antenna signals.

The at least one transmission antenna may be a helical antenna. The at least one reception antenna may be a helical antenna. In comparison with a rod antenna, use of an equally long helical antenna makes it possible to receive higher signal amplitudes for an identical electromagnetic field and to achieve a higher transmission power. In comparison with a rod antenna of comparable transmission and reception power, a helical antenna is significantly more compact, i.e. can be designed with smaller dimensions.

In one embodiment, the helical antenna includes two or four helically coiled conductors, which are arranged and connected in the manner of a dipole or turnstile dipole. A particularly high transmission or reception power may be achieved.

The transmission and reception power of an antenna, which is designed as a helical antenna, is improved by inserting a rod-shaped iron core. The higher signal amplitudes result in less error when identifying the current position of the capsule endoscope by the analysis unit. A high level of measuring accuracy may be achieved, for example, with a very small reception antenna which essentially corresponds to the dimensions of the capsule endoscope itself.

In one embodiment, a permanent magnet is assigned to the capsule endoscope. A magnetic navigation system may be used for navigation of the capsule endoscope. The magnetic navigation system may be designed in the manner described in DE 101 42 253 C1. This navigation system may include a magnet system for generating a three-dimensional gradient field. By an interaction of the permanent magnet of the capsule endoscope with the gradient field, the capsule endoscope is moved inside the body. The specification of the gradient field is effected by an input device, such as a six dimensional (6D) mouse. The capsule endoscope can be navigated intuitively inside the body. The current position of the capsule endoscope is captured continuously. It is also possible to navigate the capsule endoscope selectively to a specific position inside the body. Once there, it is possible to produce diagnostic images by an imaging system or to take tissue samples by a corresponding medical instrument, for example.

In one embodiment, the control unit is configured to process a specified desired position and to control the magnetic navigation system such that the current position of the capsule endoscope becomes the desired position. The desired position is specified in particular using an input unit, such as the 6D mouse, a computer keyboard, or similar. When moving the capsule endoscope using the magnetic navigation system, the specified desired position and the current position of the capsule endoscope are continuously compared with each other. This continuous comparison stops when the current position has become the desired position. In other words, this is a closed control loop or “closed loop”.

The capsule endoscope is limited in its movement possibilities due to the anatomical conditions of the body. A capsule endoscope which is contained in the intestine of a patient is limited in its movement by the walls of the intestine, for example. If the capsule endoscope moves against a wall, the wall offers a resistance to the capsule endoscope. The control unit then attempts to increase the magnetic field strength of the resistance in the wall direction. In order to prevent injuries to the patient, a threshold value for the magnetic field strength, above which the magnetic field strength cannot be increased, may be specified. A display unit can also be used to indicate that the threshold value has been reached. A display unit may be assigned to every possible movement direction of the 6D mouse, for example. Exceeding the magnetic field strength in a given movement direction results in an indication on the corresponding display unit. The user can then change the movement direction of the 6D mouse, such that an excess of the threshold value is no longer indicated. This helps the user when navigating the capsule endoscope through the body of the patient.

The excess of the threshold value of the magnetic field strength may be sent to the input unit as a haptic response. An input unit, in the form of a 6D mouse, may be disabled in a movement direction. As a refinement of this concept, provision can be made for making the operation of the 6D mouse in the corresponding movement direction increasingly more difficult as the threshold value is approached, until finally any movement of the 6D mouse is disabled when the threshold value is reached.

In one embodiment, the permanent magnet is designed as a bar magnet and is inserted into the reception antenna, which may be designed as a helical antenna. The permanent magnet may be accommodated in the capsule endoscope in a space-saving manner.

The problem is further solved by a method for determining the current position of a capsule endoscope inside the body of a patient. In this context, the individual embodiments of the apparatus with their advantages are correspondingly transferred to the method.

### BRIEF DESCRIPTION OF THE DRAWINGS

FIG. 1 illustrates a first embodiment of an apparatus for carrying out a minimally invasive diagnosis or intervention,

FIG. 2 illustrates a first embodiment of a capsule endoscope,

FIG. 3 illustrates a second embodiment of a apparatus for carrying out a minimally invasive diagnosis or intervention, an

FIG. 4 illustrates a second embodiment of a capsule endoscope.

### DETAILED DESCRIPTION

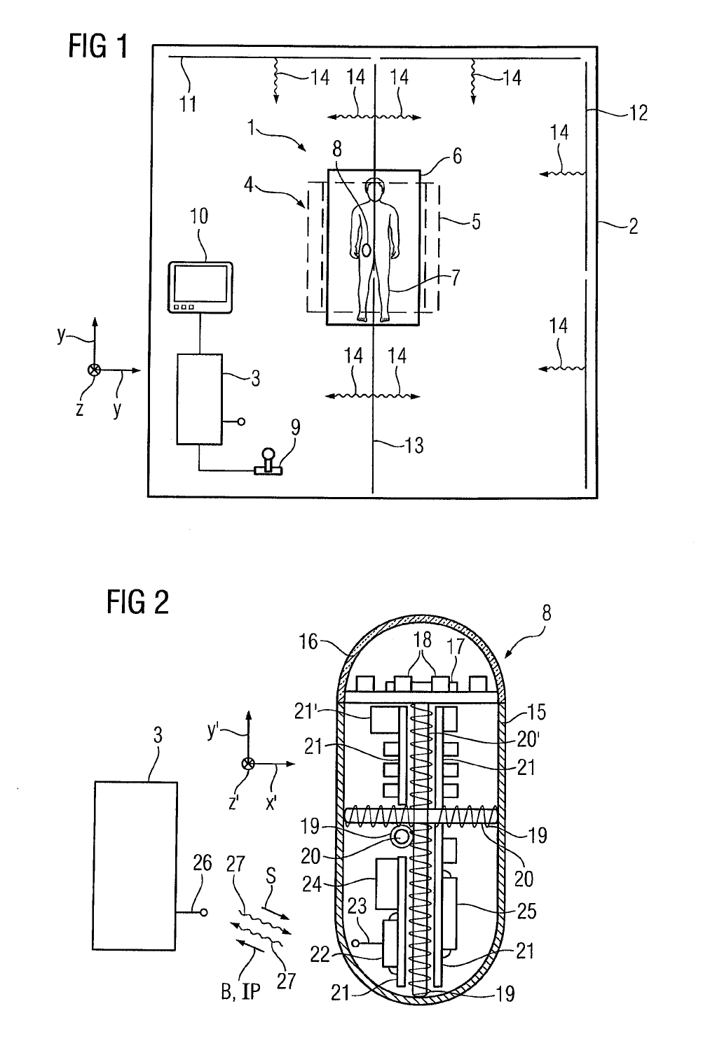

FIG. 1 illustrates a first embodiment of an apparatus (system) 1 for carrying out a minimally invasive diagnosis or intervention 1. The apparatus 1 is arranged in a treatment room 2. In FIG. 1, the treatment room 2 with the apparatus 1 is illustrated in a plan view from above. A control unit 3 is provided for controlling the apparatus 1. A magnetic navigation system 4 is controlled by the control unit 3. The magnetic navigation system 4 features a gradient coil system, which is integrated in a hollow cylindrical gantry 5. This gantry 5 essentially resembles a gantry of a magnetic resonance tomograph. The gantry 5 surrounds a patient couch (support) 6, on which a patient 7 is placed for examination. The patient 7 can be positioned in a Y direction relative to the gantry 5 using the patient couch 6.

A capsule endoscope 8 is introduced into the body of the patient 7. For an investigation of the digestive tract, the capsule endoscope 8 is swallowed by the patient 7.

The control unit 3 is used to control the magnetic navigation system 4 for navigating the capsule endoscope 8 inside the body of the patient 7. An input unit 9 in the form of a six dimensional (6D) mouse is provided for specifying a desired position SP of the capsule endoscope 8. A display unit 10 in the form of a monitor is assigned to the control unit 3 for the display of diagnostic images B.

The functionality of the magnetic navigation system 4 is described in detail in DE 101 42 253 C1.

A transmission antenna 11 running in an X-direction is attached to a first wall of the treatment room 2, for example, midway in a Z direction. A second transmission antenna 12 running in a Y direction is attached to the ceiling of the treatment room 2, for example, midway in a Y direction. A third transmission antenna 13 running in a Y direction is attached to the ceiling of the treatment room 2, for example, midway in an X direction.

The three transmission antennas 11,12,13 may be designed as helical antennas, having two helically coiled conductors in each case, which are arranged in the manner of a dipole. Both conductors are arranged one behind the other longitudinally and are separated from each other by an intermediate space. The two conductors of each helical antenna are supplied symmetrically by the control unit 3. The corresponding connection cables are not shown. The helical winding of the conductors is likewise not shown.

For the purpose of emitting electromagnetic radiation 14, the three transmission antennas 11,12,13 are controlled by the control unit 3. The control is effected such that a three-dimensional inhomogeneous electromagnetic field is produced in the treatment room 2. The electromagnetic radiation 14, which is generated by the transmission antennas 11,12,13, has a frequency of approximately 30 kHz. By virtue of the central arrangement of each of the transmission antennas 11,12,13 on a side wall or on the ceiling, uniform radiation characteristics of the respective transmission antenna 11,12,13 are achieved from the perspective of the longitudinal axis of the respective antenna.

FIG. 2 shows the capsule endoscope 8 in a sectional side view. The capsule endoscope 8 has an ellipsoidal housing 15. At the top end in a Y′ direction, the housing 15 has a transparent plastic screen 16. A medical instrument 17, such as a video camera, is arranged inside the housing 15 behind the plastic screen 16. The charge coupled device (CCD) chip 17 of the video camera is illustrated. Arranged behind the plastic screen 16 is a plurality of lighting elements 18. The lighting elements 18 are designed as light emitting diodes (LEDs). One reception antenna 19 in each case is arranged in an X′ direction, a Y′ direction and a Z′ direction in the housing 15 of the capsule endoscope 8. The orientation vectors of the three reception antennas 19 in X′, Y′ and Z′ directions are orthogonal relative to each other. Each of the reception antennas 19 are designed as a helical antenna having two helically coiled conductors which are arranged in the manner of a dipole. In FIG. 2, only the intermediate space of the reception antenna 19 running in the Y′ direction can be seen. A rod-shaped iron core 20 is inserted in each case into the helical antennas 19 running in X′ and Z′ directions. A bar magnet 20′ is inserted into the helical antenna 19 running in the Y′ direction.

The housing 15 may include a number of circuit boards 21. Various electronic components of the capsule endoscope 8 are arranged on the circuit boards 21. One such electronic component is a control unit 21′ for the conductors of each helical antenna 19. A further electronic component is the CCD chip 17 of the video camera. Another electronic component is a communication unit 22 which is equipped with a miniaturized antenna 23. A further electronic component is an energy storage unit 24 in the form of an accumulator. Finally, an analysis unit 25 is attached to a circuit board 21.

Bidirectional communication with the control unit 3 takes place via the communication unit 22 and the antenna 23. The control unit 3 is equipped with an antenna 26. A carrier frequency 27, for example, of approximately 400 MHz, is used for communication between the control unit 3 and the communication unit 22.

Diagnostic images B, which are measured using the video camera 17, may be transferred to the control unit 3 by virtue of this carrier frequency 27. The diagnostic images B may be reported on the display unit 10. Control signals S may be transferred from the control unit 3 to the capsule endoscope 8. These control signals S may be used to change the settings of the video camera 17, for example. The control signals S can also be used, for example, to switch lighting elements on or off, or to control the brightness of the lighting elements. The control signals S allow remote control of the video camera 17. It is possible to adjust the resolution, the image refresh rate, the exposure time and the camera focus of the video camera 17.

The reception antennas 19 receive the electromagnetic radiation 14, which is generated by the transmission antennas 11,12,13. The electromagnetic radiation 14 induces an antenna signal at the location of the reception antennas 19. Because the electromagnetic field which is generated by the electromagnetic radiation 14 is inhomogeneous, the radiation amplitude induced in the reception antennas 19 is dependent on location and angle. The current position IP of the capsule endoscope 8 can be calculated by the analysis unit 25. The current position IP is continuously transferred to the control unit 3 by the carrier frequency 27. The current position IP is shown on the display unit 10 at all times. A definitive assignment of the individual diagnostic images B to the current position IP of the capsule endoscope 8 is possible. The control unit 3 may be configured to store the diagnostic images B with the associated current position IP of the capsule endoscope 8.

The energy storage unit 24 is supplied with the field energy that is picked up by the reception antennas.

The inhomogeneity of the electromagnetic field may be increased by the control unit 3 switching the transmission antennas 11,12,13 on and off alternately in order to generate the electromagnetic radiation 14. Provision is made for time-multiplexed operation of the transmission antennas 11,12,13. By virtue of this switching on and off, the antenna signals, which correlate with the changing electromagnetic field, are captured consecutively in the three reception antennas 19 and analyzed as a whole by the analysis unit 25. The current position IP of the capsule endoscope 8 may be identified with a high level of accuracy.

Navigation of the capsule endoscope 8 is done by a change of the gradient field provided by the gradient coil system. The change of the gradient field changes the electromagnetic force acting on the bar magnet 20′ and moves the capsule endoscope 8 inside the body.

A desired position SP is specified by the input unit 9 for navigation. The control unit 3 continuously compares the specified desired position SP with the current position IP of the capsule endoscope 8. It controls the gradient coil system such that the current position IP of the capsule endoscope 8 ultimately becomes the desired position S. The desired position SP is set on the basis of a closed control loop.

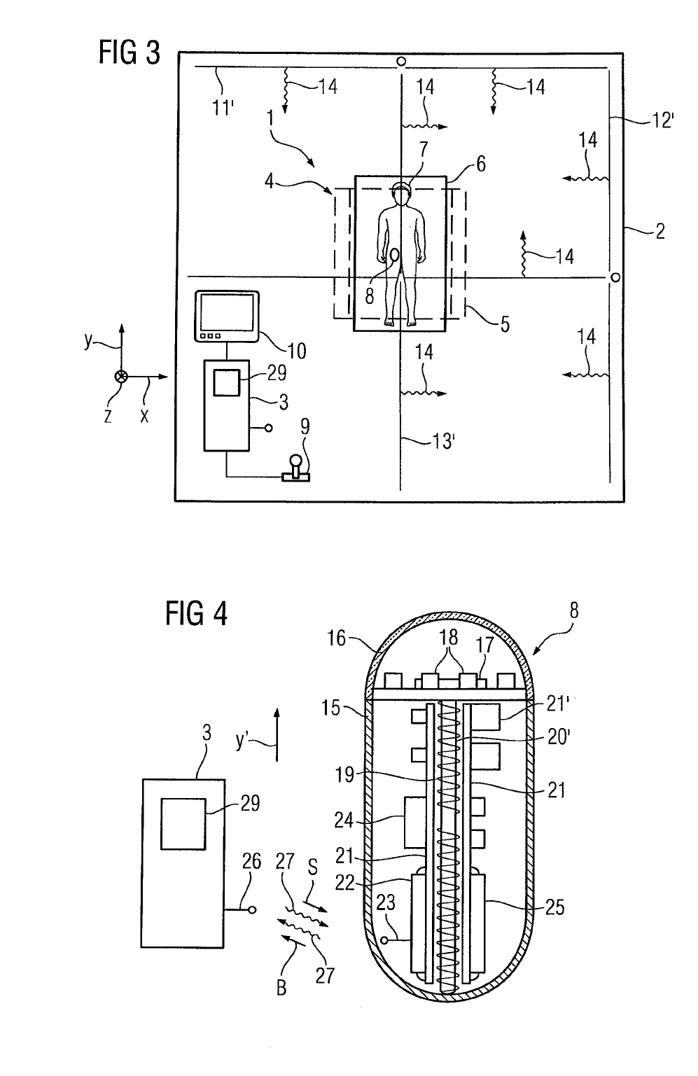

FIG. 3 shows a second embodiment of an apparatus (system) 1 for carrying out a minimally invasive diagnosis or intervention 1. This second embodiment is similar to the apparatus 1 from FIG. 1. Consequently, only the differences from this apparatus are described.

The apparatus 1 comprises three transmission antennas, which are designed as helical antennas 11′,12′,13′ and have in each case four helically formed conductors that are arranged in the manner of a turnstile aerial. Two conductors, which are arranged one behind the other and separated from each other by an intermediate space, form a limb of the helical antenna in each case. The first helical antenna 11′ is arranged on a side wall of the treatment room 2, the first limb of the first helical antenna 11′ running in an X direction and the second limb running in a Z direction. The second helical antenna 12′ is arranged on a second side wall of the treatment room 2, the first limb of the second helical antenna 12′ running in a Y direction and a second limb running in a Z direction. Finally, the third helical antenna 13′ is attached to the ceiling of the treatment room 2, the first limb of the third helical antenna 13′ running in an X direction and the second limb running in a Y direction. The four conductors of each helical antenna are supplied by the control unit 3. The corresponding connection cables and the helical winding of the conductors are not shown.

All transmission antennas 11′,12′,13′ are connected to the control unit 3. They are controlled by the control unit 3 for the purpose of emitting electromagnetic radiation 14. The control unit 3 also features an analysis unit 29, which allows analysis of antenna signals that are induced in the transmission antennas 11′,12′,13′.

A second embodiment of a capsule endoscope 8, which is illustrated in detail in FIG. 4, is introduced into the patient 7. The capsule endoscope 8 features only one reception antenna 19, which runs in a Y′ direction. Like the three reception antennas of the capsule endoscope illustrated in FIG. 2, the reception antenna 19 is designed as a helical antenna having two helically coiled conductors that are arranged in the manner of a dipole. A bar magnet 20′ is inserted into the helical antenna 19. The capsule endoscope 8 does not have an analysis unit. Instead, an analysis unit 29 is assigned to the control unit 3.

The transmission antennas 11′, 12′, 13′ generate an electromagnetic radiation 14 which results in an inhomogeneous electromagnetic field in the treatment room 2.

This electromagnetic field induces an antenna signal in the reception antenna 19. The transmission antennas 11′,12′,13′ are switched on and off alternately as in the case of the apparatus shown in FIG. 1. Multiplex operation is provided again. The reception antenna 19 is now alternately loaded and unloaded. This is achieved by virtue of the reception antenna 19 intermittently charging the energy storage unit 24. The charging of the energy storage unit 24 is an advantageous side effect in this case. The loading and unloading of the reception antenna 19 is registered by varying antenna signals at the transmission antennas 11′,12′,13′. These antenna signals are analyzed by the analysis unit 29 that is assigned to the control unit 3. The current position IP of the capsule endoscope 8 can be determined on the basis of the inhomogeneous electromagnetic field. The control unit 3 may associate diagnostic images B that were transferred using the communication unit 15 with the current position IP of the capsule endoscope 8.

The navigation of the capsule endoscope 8 by the magnetic navigation system 4 may take place analogously to the manner described for the apparatus in FIG. 1 and FIG. 2.

A method for determining the current position of a capsule endoscope inside the body of a patient may be provided. In this context, the individual embodiments of the apparatus with their advantages are correspondingly transferred to the method. In one embodiment, a method for determining a current position of a capsule endoscope inside a body of a patient is provided. The method includes emitting an electromagnetic radiation using at least one transmission antenna outside the body, receiving the electromagnetic radiation using at least one reception antenna arranged in or on the capsule endoscope, and determining, using an analysis unit, the current position of the capsule endoscope based on antenna signals that are generated by the interaction of the transmission antenna with the reception antenna.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. An apparatus for carrying out a minimally invasive diagnosis or intervention inside a body of a patient, the apparatus comprising:
a capsule endoscope that can be introduced into the body of the patient and includes at least one medical instrument,
at least one transmission antenna, which is arranged outside the body, for emitting electromagnetic radiation,
at least one reception antenna, in or on the capsule endoscope, for receiving the electromagnetic radiation, and
an analysis unit for determining the current position of the capsule endoscope using antenna signals which are generated by the interaction of the transmission antenna with the reception antenna.

2. The apparatus as claimed in claim 1, wherein the electromagnetic radiation has a frequency of approximately 30 kHz.

3. The apparatus as claimed in claim 1, wherein the capsule endoscope includes a communication unit for communicating with a control unit which is situated outside the body.

4. The apparatus as claimed in claim 3, wherein a carrier frequency of approximately 400 MHz is provided for communicating between the communication unit and the control unit.

5. The apparatus as claimed in claim 1, wherein the medical instrument is configured to determine measurement data, and that the analysis unit is configured to associate the measurement data with the current position of the capsule endoscope.

6. The apparatus as claimed in claim 1, wherein the at least one transmission antenna is configured to generate an inhomogeneous electromagnetic field.

7. The apparatus as claimed in claim 1, comprising a plurality of transmission antennas having orientation vectors that are linearly independent in relation to each other.

8. The apparatus as claimed in claim 6, wherein the transmission antennas are configured to emit the electromagnetic radiation alternately.

9. The apparatus as claimed in claim l, wherein a plurality of reception antennas having orientation vectors that are linearly independent relative to each other are arranged in the capsule endoscope.

10. The apparatus as claimed in claim 1, wherein the analysis unit is assigned to the capsule endoscope, and the communication unit is configured to send the current position, which is determined by the analysis unit, to the control unit.

11. The apparatus as claimed in claim 1, wherein the at least one reception antenna can be alternately loaded and unloaded for the purpose of generating varying antenna signals in the transmission antennas, and the analysis unit is assigned to the transmission antennas and is configured to calculate the current position of the capsule endoscope from the varying antenna signals.

12. The apparatus as claimed in claim 1, wherein the capsule endoscope includes an energy storage unit for storing the electrical energy that is transferred with the electromagnetic radiation.

13. The apparatus as claimed in claim 1, wherein the at least one transmission antenna is arranged on a wall of a treatment room including the apparatus.

14. The apparatus as claimed in claim 1, wherein the at least one transmission antenna is a helical antenna.

15. The apparatus as claimed in claim 1, wherein the at least one reception antenna is a helical antenna.

16. The apparatus as claimed in claim 1, further comprising a permanent magnet and a magnetic navigation system for navigation of the capsule endoscope.

17. The apparatus as claimed in claim 16, wherein the control unit is configured to process a specified desired position and to control the magnetic navigation system in such a way that the current position of the capsule endoscope becomes the desired position.

18. The apparatus as claimed in claim 17, wherein the control unit is configured to prohibit any further increase of the magnetic field strength if a magnetic field strength that is generated by the magnetic navigation system increases to a specified threshold value.

19. The apparatus as claimed in claim 14, wherein the permanent magnet is designed as a bar magnet and is inserted into the reception antenna, which is designed as a helical antenna.

20. A method for determining a current position of a capsule endoscope inside a body of a patient, the method comprising:
emitting an electromagnetic radiation using at least one transmission antenna outside the body,
receiving the electromagnetic radiation using at least one reception antenna arranged in or on the capsule endoscope, and
determining, using an analysis unit, the current position of the capsule endoscope based on antenna signals that are generated by the interaction of the transmission antenna with the reception antenna.

21. A capsule endoscope comprising:
a capsule housing that is operable to be introduced into the body of a patient;
a reception antenna that is arranged in or on the capsule housing, the reception antenna being operable to receive electromagnetic radiation from at least one transmission antenna outside the body of the patient; and
an analysis unit that is operable to determine the current position of the capsule housing based on antenna signals that are generated by the interaction of the reception antenna and at least one transmission antenna.
